(19) United States
(12) Patent Application Publication
(10) Pub. No.: US 2006/0014941 A1
(43) Pub. Date: Jan. 19, 2006

(54) Isolated T lymphocyte receptors specific for human autoantigens complexed with human MHC molecules and methods of making and using same

(75) Inventor: Edward F. Rosloniec, Cordova, TN (US)

Correspondence Address: JENKINS, WILSON & TAYLOR, P. A., 3100 TOWER BLVD, SUITE 1400, DURHAM, NC, 27707, US

(73) Assignee: University of Tennessee Research Foundation, Inc.

(21) Appl. No.: 11/022,040

(22) Filed: Dec. 22, 2004

Related U.S. Application Data
(60) Provisional application No. 60/532,010, filed on Dec. 22, 2003.

(51) Int. Cl. C07H 21/04; C12P 21/06; C07K 14/725

(52) U.S. Cl. 536/23.5; 530/350; 435/69.1; 435/320.1; 435/325

(57) ABSTRACT

Isolated T cell receptors having binding specificity for a particular class of MHC polypeptide in complex with autoantigens are provided. Methods of producing the isolated T cell receptors and methods of using the isolated T cell receptors in screening assays, diagnostic assays and therapeutic applications for rheumatoid arthritis are also provided. Assay kits comprising the isolated T cell receptors are further provided.

### CROSS REFERENCE TO RELATED APPLICATIONS

This application claims the benefit of U.S. Provisional Patent Application Ser. No. 60/532,010, filed Dec. 22, 2003; the disclosure of which is incorporated herein by reference in its entirety.

### GOVERNMENT INTEREST

The subject matter disclosed herein was made with U.S. Government support under Grant No. RO1-AR45201 awarded by National Institutes of Health, National Institute of Arthritis and Musculoskeletal and Skin Diseases. The U.S. Government has certain rights in the presently disclosed subject matter.

### TECHNICAL FIELD

The presently disclosed subject matter involves the identification and characterization of T cell receptor polypeptides having binding specificity for particular antigen-MHC complexes associated with the development and progression of rheumatoid arthritis. Methods of producing and using the isolated T cell receptors for research, diagnostic and therapeutic uses are also provided.

### BACKGROUND

Autoimmune diseases affect millions of people in the United States, with approximately 3-5% of the population being affected. The pathogenesis of autoimmune disease generally involves an attack by the patient's immune system on an organ or tissue, such as seen in cases of type 1 (insulin-dependent) diabetes (pancreatic β cells), multiple sclerosis (myelin basic protein), and thyroiditis (thyroglobulin or thyroid peroxidase). Certain autoimmune diseases are also characterized by systemic attacks, including immunological responses against the synovial lining, lung, and heart in rheumatoid arthritis and the skin, kidney, and heart in systemic lupus erythematosus.

Rheumatoid arthritis (RA) is a particularly devastating autoimmune disease as it affects individuals in the prime of their life and is feared because of its potential to cause chronic pain and irreversible damage of tendons, ligaments, joints, and bones. Further, it afflicts nearly 1% of the population. The symmetrical involvement of small peripheral joints has an enormous impact on hand and foot functions and poses therapeutic challenges that cannot be easily overcome by joint replacement. Also, systemic manifestations of RA are not rare and can range from relatively minor problems, such as rheumatoid nodules, to life-threatening organ disease.

In addition, RA is a systemic inflammatory disease that primarily manifests itself as synovial inflammation of diarthrodial joints. The typical histopathological changes include dense infiltration of the synovial membrane by mononuclear cells, neoangiogenesis, and hypertrophy and hyperplasia of the synovial lining.

Therapeutic management of RA has steadily improved over the last few decades, mostly due to the recognition that destruction caused by chronic inflammation is irreversible and that only early and aggressive intervention can enhance therapeutic benefit. Consequently, RA patients are now being treated early in the disease course and disease-modifying agents are widely used. Despite these successes, major challenges remain. Presently, no curative intervention is available, side effects of therapies are significant, and the disease may still progress while the patient is being treated.

The etiopathogenesis of RA is not well understood. A better understanding of the mechanism of disease progression and the type and nature of immune cell involvement is needed for continued therapeutic advances. Several lines of evidence support a central role of T lymphocytes in the disease-specific pathogenic events. However, it has heretofore proven difficult to identify with certainty the immune cells mediating the immune response and to which antigens they are responding. Therefore, a better understanding of the mechanism of origination and progression of the disease would prove beneficial in furthering therapeutic options for RA.

### SUMMARY

The presently disclosed subject matter provides in part an isolated T cell receptor polypeptide having binding specificity for an MHC polypeptide and an autoantigen bound to the MHC polypeptide. In some embodiments, the MHC polypeptide is HLA-DR4. Further, in some embodiments the autoantigen is gp39 or collagen II, and in some the autoantigen is a human gp39 or human collagen II. In some embodiments, the T cell receptor polypeptide is a heterodimeric polypeptide comprising an α chain and a β chain. In some embodiments, the α chain comprises: a polypeptide encoded by a nucleic acid sequence as set forth in any of SEQ ID NOs:1, 5 and 9; a polypeptide encoded by a nucleic acid having at least about 70% or greater sequence identity to a DNA sequence as set forth in any of SEQ ID NOs:1, 5 and 9; a polypeptide encoded by a nucleic acid capable of hybridizing under stringent conditions to a nucleic acid comprising a sequence or the complement of a sequence as set forth in any of SEQ ID NOs:1, 5 and 9; a polypeptide having an amino acid sequence of any of SEQ ID NOs:2, 6 and 10, or a biologically functional equivalent thereof; a polypeptide which is immunologically cross-reactive with antibodies which are immunologically reactive with a diversity region of a polypeptide having an amino acid sequence of any of SEQ ID NOs:2, 6 and 10; or a polypeptide comprising a fragment of a polypeptide of a), b), c), d), or e). Further, in some embodiments, the β chain comprises: a polypeptide encoded by a nucleic acid sequence as set forth in any of SEQ ID NOs:3, 7 and 11; a polypeptide encoded by a nucleic acid having at least about 70% or greater sequence identity to a DNA sequence as set forth in any of SEQ ID NOs:3, 7 and 11; a polypeptide encoded by a nucleic acid capable of hybridizing under stringent conditions to a nucleic acid comprising a sequence or the complement of a sequence as set forth in any of SEQ ID NOs:3, 7 and 11; a polypeptide having an amino acid sequence of any of SEQ ID NOs:4, 8 and 12, or a biologically functional equivalent thereof; a polypeptide which is immunologically cross-reactive with antibodies which are immunologically reactive with a diversity region of a polypeptide having an amino acid sequence of any of SEQ ID NOs:4, 8 and 12; or a polypeptide comprising a portion of a polypeptide of a), b), c), d), or e).

The presently disclosed subject matter further provides an isolated nucleic acid molecule encoding a T cell receptor polypeptide having binding specificity for an MHC polypeptide and an autoantigen bound to the MHC polypeptide. In some embodiments, the MHC polypeptide is HLA-DR4. Also, in some embodiments, the autoantigen is gp39 or collagen II, and in some, the autoantigen is a human gp39 or human collagen II. In some embodiments, the encoded polypeptide comprises an amino acid sequence of any of SEQ ID NOs:2, 4, 6, 8, 10 and 12. Further, in some embodiments, the encoded polypeptide is a heterodimeric polypeptide comprising an α chain and a β chain. Further still, in some embodiments the a chain has an amino acid sequence of any of SEQ ID NOs:2, 6 and 10, or a biologically functional equivalent thereof and the β chain has an amino acid sequence of any of SEQ ID NOs:4, 8 and 12 or a biologically functional equivalent thereof.

In yet another embodiment of the presently disclosed subject matter, a chimeric gene, comprising a novel nucleic acid molecule disclosed herein and operably linked to a heterologous promoter is provided. Further, in some embodiments, a vector comprising the chimeric gene is provided. In other embodiments, a host cell comprising the vector is provided.

In still other embodiments of the presently disclosed subject matter, an isolated antibody capable of specifically binding to a diversity region of a novel polypeptide disclosed herein is provided. In some embodiments, the antibody is capable of modulating the biological activity of a novel polypeptide disclosed herein. Further, a hybridoma cell line that produces the antibody is provided.

In still further embodiments of the presently disclosed subject matter, a method of detecting a nucleic acid molecule that encodes a T cell receptor polypeptide having binding specificity for an MHC polypeptide and an autoantigen bound to the MHC polypeptide in a biological sample containing nucleic acid material is provided. The method comprises hybridizing a novel nucleic acid molecule disclosed herein under stringent hybridization conditions to the nucleic acid material of the biological sample, thereby forming a hybridization duplex; and detecting the hybridization duplex.

In yet other embodiments of the presently disclosed subject matter, a transgenic or chimeric non-human animal is provided. The animal comprises a novel polynucleic acid disclosed herein encoding a biologically active human MHC polypeptide which is present in the genome in a copy number effective to confer expression in the animal of the human MHC polypeptide; and a human rheumatoid arthritis-associated polypeptide in an amount sufficient to induce production by the mouse of activated T cells expressing a T cell receptor with binding specificity for the human rheumatoid arthritis-associated polypeptide bound to the expressed human MHC polypeptide. In some embodiments, the human MHC polypeptide is HLA-DR4. Further, in some embodiments, the human rheumatoid arthritis-associated polypeptide is gp39 or collagen II. Still further, in some embodiments, the non-human animal is a mouse.

In other embodiments, assay kits are provided. In some embodiments, the assay kits can be used for detecting the presence of a T cell receptor polypeptide having binding specificity for an MHC polypeptide and an autoantigen bound to the MHC polypeptide in a biological sample, the kit comprising a first container containing a first antibody capable of immunoreacting with a diversity region of a novel polypeptide disclosed herein, wherein the first antibody is present in an amount sufficient to perform at least one assay. In other kit embodiments, the assay kit can be used for detecting the presence of an autoantigen bound to an HLA-DR4 polypeptide in a biological sample, the kit comprising a first container containing an isolated novel T cell receptor polypeptide disclosed herein. In still other kit embodiments, the assay kit can be used for screening compounds having binding affinity for a T cell receptor antigen-MHC binding site, the kit comprising a first container containing an isolated novel T cell receptor polypeptide disclosed herein.

In still further embodiments of the presently disclosed subject matter, a method of producing a T cell hybridoma which expresses a T cell receptor polypeptide having binding specificity for a human autoantigen bound to an HLA-DR4 polypeptide is provided. In some embodiments, the method comprises immunizing a transgenic or chimeric non-human animal with a human autoantigen, wherein the non-human animal expresses an HLA-DR4 polypeptide; isolating a T cell activated by the human autoantigen from the non-human animal; and producing a T cell hybridoma by fusing the isolated activated T cell with an immortal cell, wherein the T cell hybridoma expresses a T cell receptor polypeptide having binding specificity for the human autoantigen bound to the HLA-DR4 polypeptide. In some embodiments, the method further comprises isolating the T cell hybridoma expressing the T cell receptor having binding specificity for the human autoantigen bound to the HLA-DR4 polypeptide.

In still even further embodiments of the presently disclosed subject matter, a method of screening for candidate compounds having a binding affinity for a T cell receptor antigen-MHC binding site is provided. The method comprises providing a T cell receptor comprising an antigen-MHC binding site, wherein the antigen is a human autoantigen; contacting the T cell receptor with a test compound; measuring for binding of the test compound to the antigen-MHC binding site of the T cell receptor; and selecting the test compound as a candidate compound if the test compound binds the antigen-MHC binding site of the T cell receptor.

In yet other embodiments of the presently disclosed subject matter, a method of determining a test subject's risk for developing rheumatoid arthritis is provided. In some embodiments, the method comprises providing a T cell receptor polypeptide having binding affinity for a human autoantigen bound to an HLA-DR4 polypeptide; contacting a biological sample from a test subject with the T cell receptor polypeptide, wherein the biological sample comprises MHC polypeptides from the test subject; detecting binding of the MHC polypeptides with the T cell receptor polypeptide; and determining a test subject is at risk for developing rheumatoid arthritis if at least one of the MHC polypeptide binds the T cell receptor polypeptide.

In other embodiments of the presently disclosed subject matter, a method of inhibiting binding of a T cell receptor to an MHC polypeptide and an autoantigen bound to the MHC polypeptide in a subject is provided. The method comprises administering to a subject an isolated T cell receptor polypeptide having binding specificity for an MHC polypeptide and an autoantigen bound to the MHC polypeptide. In some embodiments, the T cell receptor polypeptide is in a pharmaceutically acceptable carrier and in some embodiments, the T cell receptor polypeptide is soluble in an aqueous solution.

In still further embodiments of the presently disclosed subject matter, a method of eliciting an immune response against a T cell receptor polypeptide having binding specificity for an MHC polypeptide and an autoantigen bound to the MHC polypeptide in a subject is provided. The method comprises in some embodiments administering to a subject a composition comprising an isolated T cell receptor polypeptide having binding specificity for an MHC polypeptide and an autoantigen bound to the MHC polypeptide. Further, in some embodiments the composition further comprises an adjuvant.

Accordingly, it is an object of the presently disclosed subject matter to provide an isolated T cell receptor polypeptide having binding specificity for a complex comprising an MHC polypeptide and an autoantigen bound to the MHC polypeptide.

An object of the presently disclosed subject matter having been stated hereinabove, and which is addressed in whole or in part by the presently disclosed subject matter, other objects will become evident as the description proceeds when taken in connection with the accompanying drawings and examples as best described hereinbelow.

### BRIEF DESCRIPTION OF THE DRAWINGS

FIG. 1 is a schematic drawing the interaction of a T lymphocyte (T cell) and an antigen presenting cell (APC) during the recognition by a TCR expressed by the T cell of an antigen presented by a major histocompatibility complex (MHC) polypeptide expressed by the APC. The TCR shown in FIG. 1 is representative of the novel TCRs of the presently disclosed subject matter, which have been isolated and cloned as disclosed herein. The novel TCRs each have binding specificity for a specific complex of a rheumatoid arthritis-associated autoantigen (CII or gp39 shown in the figure) and an MHC polypeptide presenting the autoantigen (HLA-DR4 shown in the figure).

FIG. 2 is a schematic drawing generally showing the procedure for producing a T cell hybridoma cell line. Novel T cell hybridomas disclosed herein were generated by fusing activated T cells derived from a transgenic mouse expressing a human MHC polypeptide, such as HLA-DR4, and immunized with a rheumatoid arthritis-associated autoantigen, such as for example gp39 or CII. Activated T cells were fused with an immortalized T cell lymphoma, such as for example BW5147, and stable hybridomas were selected for using selective growth media, such as for example HAT. Cell lines surviving HAT selection were screened for antigen specificity as described in the Examples and Table 1.

### BRIEF DESCRIPTION OF THE SEQUENCE LISTING

SEQ ID NOs: 1 and 2 are the nucleic acid sequences and polypeptide sequences, respectively of the α chain from an isolated TCR polypeptide expressed by a novel T cell hybridoma cell line designated DR4hCII61.

SEQ ID NOs: 3 and 4 are the nucleic acid sequences and polypeptide sequences, respectively of the β chain from an isolated TCR polypeptide expressed by a novel T cell hybridoma cell line designated DR4hCII61.

SEQ ID NOs: 5 and 6 are the nucleic acid sequences and polypeptide sequences, respectively of the α chain from an isolated TCR polypeptide expressed by a novel T cell hybridoma cell line designated DR4hCII36.

SEQ ID NOs: 7 and 8 are the nucleic acid sequences and polypeptide sequences, respectively of the β chain from an isolated TCR polypeptide expressed by a novel T cell hybridoma cell line designated DR4hCII36.

SEQ ID NOs: 9 and 10 are the nucleic acid sequences and polypeptide sequences, respectively of the α chain from an isolated TCR polypeptide expressed by a novel T cell hybridoma cell line designated DR4gp32.

SEQ ID NOs: 11 and 12 are the nucleic acid sequences and polypeptide sequences, respectively of the β chain from an isolated TCR polypeptide expressed by a novel T cell hybridoma cell line designated DR4gp32.

### DETAILED DESCRIPTION

As a disease group, autoimmune diseases comprise one of the major health problems of humankind. Rheumatoid arthritis (RA), a debilitating, relapsing autoimmune disease of the joints, alone occurs in 1% of the population. Although there are over 50 different autoimmune diseases, little is known about what initiates these diseases or what self-proteins drive the autoimmune responses.

One of the problems in studying these diseases has been the difficulty in identifying the cells mediating the autoimmune response and determining to what antigens they are responding. Toward these goals of deciphering the autoimmunity of RA and developing new biological therapeutic treatments for this autoimmune disease, the presently disclosed subject matter provides in some embodiments a transgenic mouse engineered to express a human protein, HLA-DR4, that is known to be associated with a high risk for developing RA. Further, using the transgenic mice disclosed herein, a series of novel immortal T lymphocytes (T cell hybridomas) specific for two self proteins (autoantigens) proposed to be targets of the autoimmune response in RA-type II collagen (CII) or glycoprotein 39 (gp39), have been developed and are presented herein.

The novel T cell hybridomas disclosed herein and the T cell receptors they express are unique in that they recognize human proteins suspected to be involved in autoimmunity and recognize them only when the human DR4 molecule is present. The novel T cell hybridomas are useful for a number of applications, as disclosed herein. As a non-limiting example, the novel T cell hybridomas and the cloned and expressed soluble novel TCR peptides derived from the hybridomas provide for the study of the molecular interactions that occur among these potential disease-inducing proteins. Further, the novel cells and compositions disclosed herein can be utilized for the development of novel immunotherapeutics and diagnostic tools for RA.

I. Definitions

While the following terms are believed to be well understood by one of ordinary skill in the art, the following definitions are set forth to facilitate explanation of the presently claimed subject matter.

Following long-standing patent law convention, the terms “a” and “an” mean “one or more” when used in this application, including the claims.

As used herein, the term “about,” when referring to a value or to an amount of mass, weight, time, volume, concentration or percentage is meant to encompass variations of ±20% or ±10%, in another example ±5%, in another example ±1%, and in still another example ±0.1% from the specified amount, as such variations are appropriate to perform the disclosed methods.

The term “biological sample” as used herein refers to a sample that comprises a biomolecule and/or is derived from a subject. The biological sample can be utilized for the detection of the presence and/or expression level of a gene to be determined or a polypeptide of interest. Representative biomolecules include, but are not limited to total DNA, RNA, mRNA, and polypeptides. As such, a biological sample can comprise a cell, a group of cells, fragments of cells, or cell products. Any cell, group of cells, cell fragment, or cell product can be used with the methods of the presently claimed subject matter, although cell-types and organs that would be predicted to show differential gene and/or polypeptide expression in subjects with autoimmune disease versus normal subjects are best suited. In one embodiment, the biological sample comprises blood. In one embodiment, the biological sample comprises one or more of the constituent cell types that make up blood, including but not limited to T cells, B cells, monocytes, APCs and NK/NKT cells. In another embodiment, the biological sample comprises epithelial cells, such as cheek epithelial cells. Also encompassed within the phrase “biological sample” are biomolecules that are derived from a cell or group of cells that permit gene expression levels to be determined, including but not limited to nucleic acids and polypeptides.

The term “isolated”, as used in the context of a nucleic acid molecule or polypeptide indicates that the nucleic acid molecule or polypeptide exists apart from its native environment and is not a product of nature. An isolated DNA molecule or polypeptide can exist in a purified form or can exist in a non-native environment such as a transgenic host cell.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. In one example, a p-value less than or equal to 0.05, in another example less than 0.01, in another example less than 0.005, and in yet another example less than 0.001, are regarded as significant.

As used herein, the term “T cell receptor” or “TCR” refers to a clonally distributed polypeptide expressed on the membrane surface of CD4⁺ and CD8⁺ T lymphocytes. TCRs are antigen receptors that function as a component of the immune system for recognition of peptides bound to self MHC molecules on the surface of antigen presenting cells. A TCR comprises a diversity or variable region within its polypeptide sequence that contributes to the determination of the particular antigen and MHC molecule to which the TCR has binding specificity. In turn, the specificity of a T cell for a unique antigen-MHC complex resides in the particular TCR expressed by the T cell.

The most common structural form of a TCR found in vivo is as a heterodimer of two disulfide-linked transmembrane polypeptide chains, designated α and β, each chain comprising one N-terminal diversity region, one immunoglobulin-like constant domain, a hydrophobic transmembrane region and a short cytoplasmic region. A less common type of TCR comprising γ and δ chains is found in a small subset of cells and is included by the term TCR as used herein.

Although the term TCR, as used herein, includes the entire heterodimer structure, the term is not intended to be limited to this single definition. TCR, as used herein, further includes each α and β chain individually, as well as biologically active fragments thereof, including fragments soluble in aqueous solutions, of either chain alone or both chains joined. Biologically active fragments maintain the ability to at least bind with specificity to a specific antigen, and therefore will include at least a portion of the diversity region imparting antigen-MHC complex specificity. Biologically active fragments of TCRs disclosed herein can further include other functionalities of full-length TCRs, such as forming a TCR complex with other proteins, such as signaling proteins, on the membrane surface of a T cell and activating the T cell.

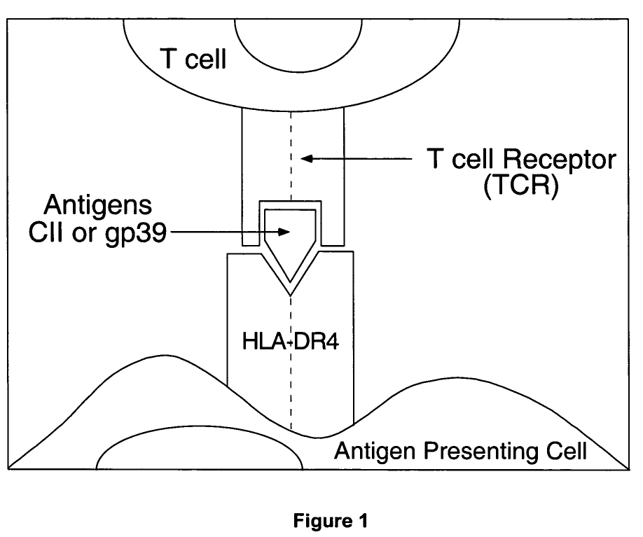

TCRs normally play a role in recognition of foreign antigens, followed by T cell activation, with a resultant activation and targeting of the immune system against the foreign antigen. FIG. 1 shows generally the interaction of a T cell and an antigen presenting cell during the recognition by a TCR of an antigen-MHC complex. However, TCRs can sometimes have specificity for and activate when contacted with MHC presented self-antigens, also referred to herein as autoantigens. Activation of T cells as a result of binding by TCRs to autoantigen-MHC complexes can play a role in certain autoimmune diseases. In particular, and as discussed in detail herein, certain rheumatoid arthritis-associated autoantigens, such as for example gp39 and collagen II (CII) have been associated with development of arthritis when in complex with particular MHC polypeptides. The presently disclosed subject matter provides isolation and characterization of particular TCRs having binding specificity for a complex of both the particular MHC polypeptide, such as for example HLA-DR4, and a rheumatoid arthritis-associated protein. The novel TCRs are useful for elucidating the role of T cell activation by autoantigens, as well as for screening, diagnostic and therapeutic methods, as disclosed herein.

The term “MHC polypeptide” and “MHC molecule” as used herein refers to a polypeptide expressed on the membrane surface of an antigen presenting cell (APC), which serves as a peptide display (antigen presentation) molecule for recognition by T cells, and more specifically, by the TCRs in complex with other signaling molecules of the T cells. MHC polypeptides can generally be categorized into two structurally distinct classes. Class I MHC molecules are present on most nucleated cells, bind and present peptides derived from cytosolic proteins, and are recognized by TCRs on CD8⁺ T cells. Class II MHC polypeptides are restricted primarily to “professional” antigen presenting cells (such as macrophages and dendritic cells), bind and present peptides derived from endocytosed proteins, and are recognized by TCRs on CD4⁺ T cells.

II. Nucleic Acids

The nucleic acid molecules employed in accordance with the presently claimed subject matter include but are not limited to isolated nucleic acid molecules encoding a T cell receptor (TCR) polypeptide having binding specificity for an MHC polypeptide and an autoantigen bound to the MHC polypeptide. In some embodiments, nucleic acid molecules of the presently disclosed subject matter include nucleic acid molecules having sequences of any one of odd-numbered SEQ ID NOs: 1-11; sequences substantially identical to sequences of any one of odd-numbered SEQ ID NOs: 1-11; conservative variants thereof, subsequences and elongated sequences thereof, complementary DNA molecules, and corresponding RNA molecules. The presently claimed subject matter also encompasses genes, cDNAs, chimeric genes, and vectors comprising disclosed nucleic acid sequences. In some embodiments, the isolated nucleic acid molecule encodes a TCR having binding specificity for an HLA-DR4 MHC molecule and a rheumatoid arthritis-associated autoantigen, such as, for example gp39 and collagen II. In some embodiments of the presently disclosed subject matter, the isolated nucleic acid molecules encode a polypeptide of any of even-numbered SEQ ID NOs: 2-12.

The presently disclosed subject matter further includes methods of detecting nucleic acid molecules having binding specificity for MHC polypeptides and autoantigens bound to the MHC polypeptides in biological samples. In an embodiment, the method comprises hybridizing a novel nucleic acid molecule described herein under stringent hybridization conditions to a complementary nucleic acid molecule present in the biological sample to form a hybridization duplex and then detecting the hybridization duplex.

The term “nucleic acid molecule” refers to deoxyribonucleotides or ribonucleotides and polymers thereof in either single- or double-stranded form. Unless specifically limited, the term encompasses nucleic acids containing known analogues of natural nucleotides that have similar properties as the reference natural nucleic acid. Unless otherwise indicated, a particular nucleotide sequence also implicitly encompasses conservatively modified variants thereof (e.g., degenerate codon substitutions), complementary sequences, subsequences, elongated sequences, as well as the sequence explicitly indicated. The terms “polynucleotide”, “nucleic acid molecule” or “nucleotide sequence” can also be used in place of “gene”, “cDNA”, or “mRNA”. Nucleic acids can be derived from any source, including any organism.

The term “substantially identical”, in the context of two nucleotide sequences, refers to two or more sequences or subsequences that in one example have at least 60%, in another example about 70%, in another example about 80%, in another example about 90-95%, and in yet another example about 99% nucleotide identity, when compared and aligned for maximum correspondence, as measured using one of the following sequence comparison algorithms or by visual inspection. In one example, the substantial identity exists in nucleotide sequences of at least 50 residues, in another example in nucleotide sequence of at least about 100 residues, in another example in nucleotide sequences of at least about 150 residues, and in yet another example in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “target” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in one embodiment at least an about 14 to 40 nucleotide sequence of a nucleic acid molecule of the presently claimed subject matter. In one example, probes comprise 14 to 20 nucleotides, or even longer where desired, such as 30, 40, 50, 60, 100, 200, 300, or 500 nucleotides or up to the full length of any of those set forth as odd-numbered SEQ ID NOs: 1-11. Such fragments can be readily prepared by, for example, directly synthesizing the fragment by chemical synthesis, by application of nucleic acid amplification technology, or by introducing selected sequences into recombinant vectors for recombinant production.

The phrase “hybridizing to” refers to complementary hybridization between a probe nucleic acid molecule and a target nucleic acid molecule to form a hybridization duplex and embraces minor mismatches that can be accommodated by reducing the stringency of the hybridization media to achieve the desired hybridization. “Hybridizing under stringent wash conditions” refers to a hybridization which occurs only if high identity occurs between complementary sequences.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tₘ) for the specific sequence at a defined ionic strength and pH. Typically, under “stringent conditions” a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC, SM NaCl at 65° C. An example of stringent wash conditions is 15 minutes in 0.2×SSC buffer at 65° C. (see Sambrook and Russell, 2001 for a description of SSC buffer). Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently claimed subject matter: a probe nucleotide sequence hybridizes in one example to a target nucleotide sequence in 7% sodium dodecyl sulfate (SDS), 0.5M NaPO₄, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO₄, 1 mm EDTA at 50° C. followed by washing in 0.1×SSC, 0.1% SDS at 50° C.; in yet another example, a probe and target sequence hybridize in 7% sodium dodecyl sulfate (SDS), 0.5M NaPO₄, 1 mm EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, are biologically functional equivalents, and/or are immunologically cross-reactive. These terms are defined further under Section III having the heading “Polypeptides” herein below. Nucleic acid molecules that do not hybridize to each other under stringent conditions are still substantially identical if the corresponding proteins are substantially identical. This can occur, for example, when two nucleotide sequences are significantly degenerate as permitted by the genetic code.

The term “conservatively substituted variants” refers to nucleic acid sequences having degenerate codon substitutions wherein the third position of one or more selected (or all) codons is substituted with mixed-base and/or deoxyinosine residues (Ohtsuka et al., 1985; Batzer et al., 1991; Rossolini et al., 1994)

The term “subsequence” refers to a sequence of nucleic acids that comprises a part of a longer nucleic acid sequence. An exemplary subsequence is a probe, described herein above, or a primer. The term “primer” as used herein refers to a contiguous sequence comprising in one example about 8 or more deoxyribonucleotides or ribonucleotides, in another example 10-20 nucleotides, and in yet another example 20-30 nucleotides of a selected nucleic acid molecule. The primers of the presently claimed subject matter encompass oligonucleotides of sufficient length and appropriate sequence so as to provide initiation of polymerization on a nucleic acid molecule of the presently claimed subject matter.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The term “complementary sequences”, as used herein, indicates two nucleotide sequences that comprise antiparallel nucleotide sequences capable of pairing with one another upon formation of hydrogen bonds between base pairs. As used herein, the term “complementary sequences” means nucleotide sequences which are substantially complementary, as can be assessed by the same nucleotide comparison set forth above, or is defined as being capable of hybridizing to the nucleic acid segment in question under relatively stringent conditions such as those described herein. A particular example of a complementary nucleic acid segment is an antisense oligonucleotide.

The term “gene” refers broadly to any segment of DNA associated with a biological function. A gene encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “gene expression” generally refers to the cellular processes by which a biologically active polypeptide is produced from a DNA sequence.

The presently claimed subject matter can also employ chimeric genes. The term “chimeric gene”, as used herein, refers to a heterologous promoter region operatively linked to a nucleotide sequence encoding a therapeutic polypeptide; a nucleotide sequence producing an antisense RNA molecule; a RNA molecule having tertiary structure, such as a hairpin structure; or a double-stranded RNA molecule. A “heterologous” promoter is a promoter not normally found in nature operably linked to the nucleotide sequence in the chimeric gene. In some embodiments, the presently disclosed subject matter includes a chimeric gene comprising a nucleic acid molecule encoding a TCR polypeptide having binding specificity for an MHC polypeptide and an autoantigen bound to the MHC polypeptide, operably linked to a heterologous promoter.

The terms “operatively linked” and “operably linked”, as used herein, refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “construct” as used herein means a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence of a same gene and functions to direct transcription of the coding sequence. The promoter region comprises a transcriptional start site, and can additionally include one or more transcriptional regulatory elements. The promoter can be a minimal promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human b-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the β-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include the PSA promoter (Yu et al., 1999; Lee et al., 2000), the probasin promoter (Greenberg et al., 1994; Yu et al., 1999), and the MUC1 promoter (Kurihara et al., 2000) as discussed above, as well as other tissue-specific and cell-type specific promoters known to those of skill in the art.

Inducible promoters are also encompassed by the term “promoter” as used herein. An “inducible” promoter is one for which the transcription level of an operably linked gene varies based on the presence of a certain stimulus. Genes that are under the control of inducible promoters are expressed only, or to a greater degree, in the presence of an inducing agent, (e.g., transcription under control of the metallothionein promoter is greatly increased in presence of certain metal ions). Inducible promoters include transcriptional regulatory elements (TREs), which stimulate transcription when their inducing factors are bound. For example, there are TREs for serum factors, steroid hormones, retinoic acid and cyclic AMP. Promoters containing a particular TRE can be chosen in order to obtain an inducible response, and in some cases, the TRE itself can be attached to a different promoter, thereby conferring inducibility to the recombinant gene.

When used in the context of a promoter, the term “linked” as used herein refers to a physical proximity of promoter elements such that they function together to direct transcription of an operably linked nucleotide sequence.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook, 1990 and PCT International Publication No. WO 97/47763. Exemplary reporter genes for transcriptional analyses include the lacZ gene (see e.g., Rose & Botstein, 1983), Green Fluorescent Protein (GFP; Cubitt et al., 1995), luciferase, and chloramphenicol acetyl transferase (CAT). Reporter genes for methods to produce transgenic animals include but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently claimed subject matter.

An amount of reporter gene can be assayed by any method for qualitatively or quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, in one example an increase by about 2-fold or greater relative to a control measurement, in another example an increase by about 5-fold or greater, and in yet another example an increase by about 10-fold or greater.

The presently claimed subject matter further includes a vector comprising a nucleic acid molecule encoding a TCR polypeptide having specificity for an MHC polypeptide and an autoantigen bound to the MHC polypeptide. The term “vector”, as used herein refers to a DNA molecule having sequences that enable the transfer of those sequences to a compatible host cell. A vector also includes nucleotide sequences to permit ligation of nucleotide sequences within the vector, wherein such nucleotide sequences are also replicated in a compatible host cell. A vector can also mediate recombinant production of a therapeutic polypeptide, as described further herein below.

Nucleic acids of the presently claimed subject matter can be cloned, synthesized, recombinantly altered, mutagenized, or combinations thereof. Standard recombinant DNA and molecular cloning techniques used to isolate nucleic acids are known in the art. Exemplary, non-limiting methods are described by Silhavy et al., 1984; Ausubel et al., 1992; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

III. Polypeptides

The presently disclosed subject matter provides novel isolated T cell receptor (TCR) polypeptides. The novel TCR polypeptides each have binding specificity for an MHC polypeptide and an autoantigen bound to the MHC polypeptide. In some embodiments, the MHC polypeptide is an HLA-DR4 MHC polypeptide. In some embodiments, the autoantigen is a rheumatoid arthritis-associated peptide, for example, gp39 or collagen II. In some embodiments, the novel TCR polypeptide is a heterodimeric polypeptide. That is, a polypeptide having two polypeptide subunits bound together with each subunit having differing polypeptide sequences. The heterodimeric polypeptide can comprise α chain subunit and a β chain subunit. In some embodiments, the α chain subunit can comprise a polypeptide encoded by a nucleic acid sequence as set forth in any of SEQ ID NOs:1, 5 and 9; a polypeptide encoded by a nucleic acid having at least about 70% or greater sequence identity to a DNA sequence as set forth in any of SEQ ID NOs:1, 5 and 9; a polypeptide encoded by a nucleic acid capable of hybridizing under stringent conditions (as defined in Section II above) to a nucleic acid comprising a sequence or the complement of a sequence as set forth in any of SEQ ID NOs:1, 5 and 9; a polypeptide having an amino acid sequence of any of SEQ ID NOs:2, 6 and 10, or a biologically functional equivalent thereof; a polypeptide which is immunologically cross-reactive with antibodies which are immunologically reactive with a diversity region of a polypeptide having an amino acid sequence of any of SEQ ID NOs:2, 6 and, 10; or a polypeptide comprising a fragment of one of the preceding polypeptides. Further, in some embodiments, the β chain subunit can comprise a polypeptide encoded by a nucleic acid sequence as set forth in any of SEQ ID NOs:3, 7 and 11; a polypeptide encoded by a nucleic acid having at least about 70% or greater sequence identity to a DNA sequence as set forth in any of SEQ ID NOs:3, 7 and 11; a polypeptide encoded by a nucleic acid capable of hybridizing under stringent conditions (as defined in Section II above) to a nucleic acid comprising a sequence or the complement of a sequence as set forth in any of SEQ ID NOs:3, 7 and 11; a polypeptide having an amino acid sequence of any of SEQ ID NOs:4, 8 and 12, or a biologically functional equivalent thereof; a polypeptide which is immunologically cross-reactive with antibodies which are immunologically reactive with a diversity region of a polypeptide having an amino acid sequence of any of SEQ ID NOs:4, 8 and 12; or a polypeptide comprising a portion of the preceding polypeptides.

The polypeptides employed in accordance with the presently claimed subject matter include but are not limited to polypeptides as defined herein above; a polypeptide substantially identical to a polypeptide as defined herein above; a polypeptide fragment of a polypeptide as defined herein above (in one embodiment, biologically functional fragments); fusion proteins comprising a polypeptide as defined herein above; biologically functional analogs thereof; and polypeptides that cross-react with an antibody that specifically recognizes a therapeutic polypeptide as defined herein below. The polypeptides employed in accordance with the presently claimed subject matter include but are not limited to isolated polypeptides, polypeptide fragments, fusion proteins comprising the disclosed amino acid sequences, biologically functional analogs, and polypeptides that cross-react with an antibody that specifically recognizes a disclosed polypeptide.

The term “isolated”, as used in the context of a polypeptide, indicates that the polypeptide exists apart from its native environment and is not a product of nature. An isolated polypeptide can exist in a purified form or can exist in a non-native environment such as, for example, in a transgenic host cell.

The term “substantially identical” in the context of two or more polypeptide sequences is measured as polypeptide sequences having in one example about 35%, or 45%, in another example from 45-55%, and in another example 55-65% of identical or functionally equivalent amino acids. In another example, two or more “substantially identical” polypeptide sequences will have about 70%, or in another example about 80%, in another example about 90%, in another example about 95%, and in yet another example about 96%, 97%, 98% or 99% identical or functionally equivalent amino acids. Percent “identity” and methods for determining identity are defined herein below in Section IV under the heading “Nucleotide and Amino Acid Sequence Comparisons”.

Substantially identical polypeptides also encompass two or more polypeptides sharing a conserved three-dimensional structure. Computational methods can be used to compare structural representations, and structural models can be generated and easily tuned to identify similarities around important active sites or ligand binding sites (see Barton, 1998; Saqi et al., 1999; Henikoff et al., 2000; Huang et al., 2000).

The terms “biologically functional equivalent” and “functionally equivalent” in the context of amino acid sequences are known in the art and are based on the relative similarity of the amino acid side-chain substituents (see Henikoff & Henikoff, 2000). Relevant factors for consideration include side-chain hydrophobicity, hydrophilicity, charge, and size. For example, arginine, lysine, and histidine are all positively charged residues; alanine, glycine, and serine are all of similar size; and phenylalanine, tryptophan, and tyrosine all have a generally similar shape. By this analysis, described further herein below, arginine, lysine, and histidine; alanine, glycine, and serine; and phenylalanine, tryptophan, and tyrosine; are defined herein as biologically functional equivalents.

In making biologically functional equivalent amino acid substitutions, the hydropathic index of amino acids can be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte & Doolittle, 1982). It is known that certain amino acids can be substituted for other amino acids having a similar hydropathic index or score and still retain a similar biological activity. In making changes based upon the hydropathic index, the substitution of amino acids involves in one example those with hydropathic indices within ±2 of the original value, in another example those within ±1 of the original value, and in yet another example those within ±0.5 of the original value.

It is also understood in the art that the substitution of like amino acids can be made effectively on the basis of hydrophilicity. U.S. Pat. No. 4,554,101, incorporated herein by reference in its entirety, states that the greatest local average hydrophilicity of a protein, as governed by the hydrophilicity of its adjacent amino acids, correlates with its immunogenicity and antigenicity, e.g., with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids is in one example those with hydrophilicity values within ±2 of the original value, in another example those within ±1 of the original value, and in yet another example those within ±0.5 of the original value.

The methods of the presently claimed subject matter can also employ polypeptide fragments or functional portions of a polypeptide. Such functional portions need not comprise all or substantially all of the amino acid sequence of a native gene product. The term “functional” includes any biological activity or feature of the polypeptide. In the case of a TCR polypeptide, the biological activity is, for example, an ability to recognize and bind to specific complexes of antigens bound to MHC polypeptides. Specifically with regard to some embodiments of the presently disclosed subject matter, the novel TCR polypeptides and functional fragments or portions thereof can have the biological activity of binding specifically to a complex of an HLA-DR4 MHC polypeptide and an autoantigen bound to the HLA-DR4 polypeptide. In some embodiments, the autoantigen is a rheumatoid arthritis-associated peptide, such as for example gp39 or collagen II.

The presently claimed subject matter also includes longer sequences of a polypeptide. For example, one or more amino acids can be added to the N-terminus or C-terminus of the novel polypeptide. Fusion proteins comprising polypeptide sequences are also provided within the scope of the presently claimed subject matter. Methods of preparing such proteins are known in the art. In one example, the fusion protein includes any biological activity of a novel polypeptide disclosed herein. In the case of a TCR polypeptide, the biological activity is in one embodiment any biological activity of a native TCR polypeptide, for example, a binding specificity for an MHC polypeptide and an autoantigen bound to the MHC polypeptide, as disclosed herein. Optionally, a fusion protein can have additional biological activities provided by the fused heterologous sequence.

The presently claimed subject matter also encompasses functional analogs of a therapeutic polypeptide. Functional analogs share at least one biological function with a polypeptide. In the context of amino acid sequence, biologically functional analogs, as used herein, are peptides in which certain, but not most or all, of the amino acids can be substituted. Functional analogs can be created at the level of the corresponding nucleic acid molecule, altering such sequence to encode desired amino acid changes. In one embodiment, changes can be introduced to improve a biological function of the polypeptide, e.g., a binding specificity for an MHC polypeptide and an autoantigen bound to the MHC polypeptide.

The presently claimed subject matter also encompasses recombinant production of the disclosed polypeptides. Briefly, a nucleic acid sequence encoding a novel polypeptide disclosed herein, is cloned into a construct, the construct is introduced into a host organism, where it is recombinantly produced.

The term “host organism” refers to any organism into which a disclosed vector has been introduced. In one embodiment, the host organism is a bacteria or yeast. In another embodiment, it is a warm-blooded vertebrate, and in another embodiment, a mammal.

IV. Nucleotide and Amino Acid Sequence Comparisons

The terms “identical” or percent “identity” in the context of two or more nucleotide or polypeptide sequences, refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical” in regards to a nucleotide or polypeptide sequence means that a particular sequence varies from the sequence of a naturally occurring sequence by one or more deletions, substitutions, or additions, the net effect of which is to retain at least some of biological activity of the natural gene, gene product, or sequence. Such sequences include “mutant” sequences, or sequences wherein the biological activity is altered to some degree but retains at least some of the original biological activity. The term “naturally occurring”, as used herein, is used to describe a composition that can be found in nature as distinct from being artificially produced by man. For example, a protein or nucleotide sequence present in an organism, which can be isolated from a source in nature and which has not been intentionally modified by man in the laboratory, is naturally occurring.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman (1981), by the homology alignment algorithm of Needleman & Wunsch (1970), by the search for similarity method of Pearson & Lipman (1988), by computerized implementations of these algorithms (GAP, BESTFIT, FASTA, and TFASTA in the GCG® WISCONSIN PACKAGE®, available from Accelrys, Inc., San Diego, Calif., United States of America), or by visual inspection (see generally, Ausubel et al., 1992).

An exemplary algorithm for determining percent sequence identity and sequence similarity is the BLAST algorithm, which is described in Altschul et al., 1990. Software for performing BLAST analyses is publicly available through the National Center for Biotechnology Information (available at the NCBI web site). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1992).

In addition to calculating percent sequence identity, the BLAST algorithm also performs a statistical analysis of the similarity between two sequences (see e.g., Karlin & Altschul, 1993). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1 in one example, less than about 0.01 in another example, and less than about 0.001 in yet another example.

V. Generation of Antibodies and Hybridoma Cell Lines

V.A. Antibodies and B Cell Hybridomas

In still another embodiment, the presently disclosed subject matter provides an antibody immunoreactive with a novel polypeptide disclosed herein. In some embodiments, an antibody capable of specifically binding to a diversity region of a TCR polypeptide having binding specificity for an MHC polypeptide and an autoantigen bound to the MHC polypeptide. In some embodiments, the MHC polypeptide is an HLA-DR4 polypeptide and the autoantigen is a rheumatoid arthritis-associated polypeptide, such as for example gp39 or human collagen II. Preferably, an antibody of the presently disclosed subject matter is a monoclonal antibody. In some embodiments, the antibodies of the presently disclosed subject matter are capable of modulating the biological activity of a novel polypeptide disclosed herein. In particular, the antibody, upon binding the epitope within the diversity region of a TCR polypeptide disclosed herein, modulates the capability of the TCR polypeptide ability to recognize and bind to a specific complex of an antigen bound to an MHC polypeptide. In some embodiments, the antibody can inhibit the biological activity of the TCR polypeptide to which it binds.

Approaches for preparing and characterizing antibodies are well known in the art (See, e.g., Antibodies A Laboratory Manual, E. Howell and D. Lane, Cold Spring Harbor Laboratory, 1988). Briefly, a polyclonal antibody is prepared by immunizing an animal with an immunogen comprising a polypeptide or polynucleotide of the presently disclosed subject matter, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the presently disclosed subject matter) with a carrier. Exemplary carriers are keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier protein are well known in the art and include glutaraldehyde, m-maleimidobencoyl-N-hydroxysuccinimide ester, carbodiimide and bis-biazotized benzidine.

As is also well known in the art, immunogencity to a particular immunogen can be enhanced by the use of non-specific stimulators of the immune response known as adjuvants. Exemplary adjuvants include complete Freund's adjuvant, incomplete Freund's adjuvant and aluminum hydroxide adjuvant.

The amount of immunogen used for the production of polyclonal antibodies varies, inter alia, upon the nature of the immunogen as well as the animal used for immunization. A variety of routes can be used to administer the immunogen, e.g. subcutaneous, intramuscular, intradermal, intravenous and intraperitoneal. The production of polyclonal antibodies is monitored by sampling blood of the immunized animal at various points following immunization. When a desired level of immunogenicity is obtained, the immunized animal can be bled and the serum isolated and stored.

A monoclonal antibody specific for a polypeptide disclosed herein can be readily prepared through use of well-known techniques such as those exemplified in U.S. Pat. No. 4,196,265, herein incorporated by reference. Typically, a technique involves first immunizing a suitable animal with a selected antigen (e.g., a polypeptide or polynucleotide of the presently disclosed subject matter) in a manner sufficient to provide an immune response. Rodents such as mice and rats are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, for example, by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of B cell hybridomas from which specific hybridomas are selected. Typically, selection of hybridomas is performed by culturing the cells by single-clone dilution in microtiter plates, followed by testing the individual clonal supernatants for reactivity with an antigen-polypeptides. The selected clones can then be propagated indefinitely to provide the monoclonal antibody of interest.

By way of specific example, to produce an antibody and B cell hybridoma cell line producing the antibody of the presently disclosed subject matter, mice are injected intraperitoneally with between about 1-200 μg of an antigen comprising a polypeptide disclosed herein. B lymphocyte cells are stimulated to grow by injecting the antigen in association with an adjuvant such as complete Freund's adjuvant (a non-specific stimulator of the immune response containing killed Mycobacterium tuberculosis). At some time (e.g., at least two weeks) after the first injection, mice are boosted by injection with a second dose of the antigen mixed with incomplete Freund's adjuvant.

A few weeks after the second injection, mice are tail bled and the sera titered by immunoprecipitation against radiolabeled antigen. Preferably, the method of boosting and titering is repeated until a suitable titer is achieved. The spleen of the mouse with the highest titer is removed and the spleen lymphocytes are obtained by homogenizing the spleen with a syringe. Typically, a spleen from an immunized mouse contains approximately 5×10⁷ to 2×10⁸ lymphocytes.

Mutant lymphocyte cells known as myeloma cells are obtained from laboratory animals in which such cells have been induced to grow by a variety of well-known methods. Myeloma cells lack the salvage pathway of nucleotide biosynthesis. Because myeloma cells are tumor cells, they can be propagated indefinitely in tissue culture, and are thus denominated immortal. Numerous cultured cell lines of myeloma cells from mice and rats, such as murine NS-1 myeloma cells, have been established.

Myeloma cells are combined under conditions appropriate to foster fusion with the normal antibody-producing cells from the spleen of the mouse or rat injected with the antigen/polypeptide of the subject matter disclosed herein. Fusion conditions include, for example, the presence of polyethylene glycol. The resulting fused cells are hybridoma cells. Like myeloma cells, hybridoma cells grow indefinitely in culture.

Hybridoma cells are separated from unfused myeloma cells by culturing in a selection medium such as HAT media (hypoxanthine, aminopterin, thymidine). Unfused myeloma cells lack the enzymes necessary to synthesize nucleotides from the salvage pathway because they are killed in the presence of aminopterin, methotrexate, or azaserine. Unfused lymphocytes also do not continue to grow in tissue culture. Thus, only cells that have successfully fused (hybridoma cells) can grow in the selection media.

Each of the surviving hybridoma cells produces a single antibody. These cells are then screened for the production of the specific antibody immunoreactive with an antigen/polypeptide of the presently disclosed subject matter. Single cell hybridomas are isolated by limiting dilutions of the hybridomas. The hybridomas are serially diluted many times and, after the dilutions are allowed to grow, the supernatant is tested for the presence of the monoclonal antibody. The clones producing that antibody are then cultured in large amounts to produce an antibody of the presently disclosed subject matter in convenient quantity.

By use of a monoclonal antibody of the presently disclosed subject matter, specific polypeptides and polynucleotide disclosed herein can be recognized as antigens, and thus identified. Once identified, those polypeptides and polynucleotide can be isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

V.B. T Cell Hybridomas

The presently disclosed subject matter also provides T cell hybridoma cell lines which can produce the novel TCR polypeptides disclosed herein having binding specificity for an MHC polypeptide and an autoantigen bound to the MHC polypeptide. Each T cell hybridoma produces a specific TCR polypeptide having a unique diversity region and which is specific for a particular class of MHC polypeptide bound in complex to a specific antigen. In some embodiments, the MHC polypeptide is an HLA-DR4 MHC polypeptide and the autoantigen is a rheumatoid arthritis-associated peptide, such as for example gp39 or collagen II.

The presently disclosed subject matter further provides methods of producing the novel T cell hybridomas. In some embodiments, the methods comprise producing the novel T cell hybridomas by immunizing a transgenic or chimeric non-human animal with a human autoantigen, wherein the non-human animal expresses an HLA-DR4 polypeptide; isolating a T cell activated by the human autoantigen from the non-human animal; and producing a T cell hybridoma by fusing the isolated activated T cell with an immortal cell, wherein the T cell hybridoma expresses a T cell receptor polypeptide having binding specificity for the human autoantigen bound to the HLA-DR4 polypeptide.

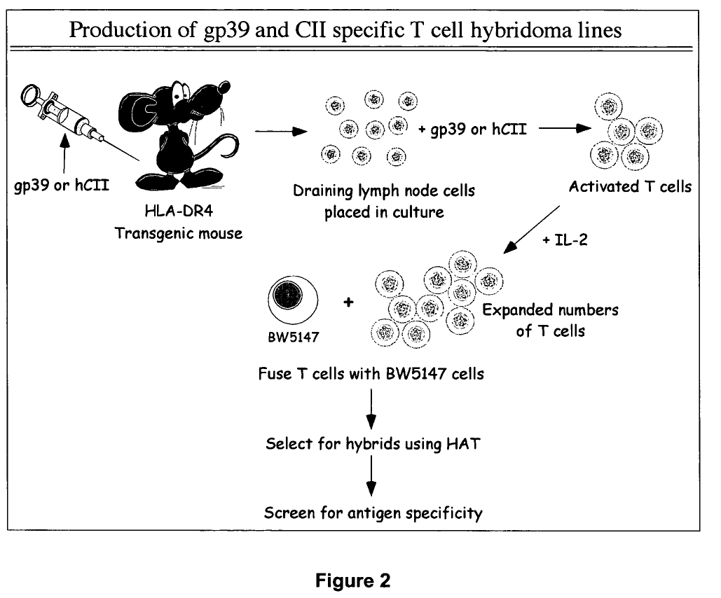

An exemplary application of the method described above for producing a T cell hybridoma is shown in FIG. 2. A transgenic animal expressing a foreign MHC polypeptide is immunized with an autoantigen, such as a rheumatoid arthritis-associated autoantigen, including but not limited to collagen II or gp39. In some embodiments, the transgenic mouse expresses human HLA-DR4 as the foreign MHC polypeptide. As shown in FIG. 2, the transgenic animal can be immunized by injection of the autoantigen into the animal, with or without one or more adjuvants, as is known in the art. Immunization with the antigen induces an immune response mounted by the immunized animal, resulting in part in activation of T cells specific for the antigen. The activated T cells can be isolated from the animal, for example by removing and culturing lymph tissue.

The activated T cells are cultured and the culture expanded through stimulation with cytokines, such as IL-2, and other agents, as is generally known in the art. T cell hybridomas are created by fusion of the expanded T cells with complementary immortalized cells, and generated hybridomas selected using similar techniques as described in Section V.A. for the production of B cell hybridomas. However, one difference between the methods is in the immortalized cell line chosen for fusion with the T cells. For producing T cell hybridomas, an immortalized T cell lymphoma can be used. For example, in some embodiments of the presently disclosed subject matter a strain of BW5147 cells (e.g. ATCC No. CRL-1588, American Type Culture Collection, Manassas, Va., U.S.A.) is used for fusion with the T cells.

The broad sampling of T cell hybridomas produced is then screened for particular hybridomas expressing TCRs having specificity for the MHC polypeptide expressed by the transgenic animal in combination with the autoantigen with which the transgenic animal was immunized. Selected hybridomas can then be isolated and cultured for further analysis and used, as disclosed herein, including production and isolation of the selected TCRs for sequencing, cloning, and further characterization and use.

VI. Transgenic Animals

It is within the scope of the presently disclosed subject matter to prepare a transgenic non-human animal that expresses an MHC polypeptide derived from a different species, for example a human. Further, the transgenic animal can have introduced into it a rheumatoid arthritis-associated polypeptide in an amount sufficient to induce production by the mouse of activated T cells expressing a T cell receptor with binding specificity for the rheumatoid arthritis-associated polypeptide bound to the expressed xenogenic MHC polypeptide. In some embodiments, the MHC polypeptide is a human HLA-DR4 polypeptide. In some embodiments, the rheumatoid arthritis-associated polypeptide can be gp39 or collagen II. Further, the TCR polypeptide expressed by the transgenic animal can comprise the novel TCR polypeptides disclosed herein. In some embodiments, the transgenic animal is a mouse.

Techniques for the preparation of transgenic animals are known in the art. Exemplary techniques are described in U.S. Pat. No. 5,489,742 (transgenic rats); U.S. Pat. Nos. 4,736,866; 5,550,316; 5,614,396; 5,625,125; and 5,648,061 (transgenic mice); U.S. Pat. Nos. 5,573,933 (transgenic pigs); 5,162,215 (transgenic avian species) and U.S. Pat. No. 5,741,957 (transgenic bovine species), the entire contents of each of which are herein incorporated by reference.

With respect to a representative method for the preparation of a transgenic mouse, cloned recombinant or synthetic DNA sequences or DNA segments encoding an MHC polypeptide gene product from a different species are injected into fertilized mouse eggs. The injected eggs are implanted in pseudo pregnant females and are grown to term to provide transgenic mice whose cells express the foreign MHC polypeptide.

VII. Compositions of Matter

In an embodiment of the presently disclosed subject matter, a composition of matter comprising a polypeptide or polynucleotide disclosed herein and a physiologically acceptable carrier is provided. In some embodiments, the composition comprises an isolated TCR polypeptide having binding specificity for an MHC polypeptide and an autoantigen bound to the MHC polypeptide and a carrier. In some embodiments, for example, the TCR polypeptide can be a polypeptide of any of even-numbered SEQ ID NOs: 2-12, a polypeptide encoded by a polynucleic acid having a DNA sequence set forth in any of odd-numbered SEQ ID NOs: 1-11, fragments thereof or functional equivalents thereof.

A composition of the presently disclosed subject matter can typically be administered parenterally in dosage unit formulations containing standard, well-known nontoxic physiologically acceptable carriers and adjuvants as desired. The term “parenteral” as used herein includes intravenous, intramuscular, intra-arterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable carriers and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Exemplary carriers include neutral saline solutions buffered with phosphate, lactate, Tris, and the like. Of course, one purifies the carrier sufficiently to render it essentially free of undesirable contaminants, such as endotoxins and other pyrogens such that it does not cause any untoward reactions in the subject receiving the composition.

VIII. Assay Kits

In another aspect, the presently disclosed subject matter provides assay kits for detecting the presence of a novel polypeptide disclosed herein, namely a TCR having binding specificity for an MHC polypeptide and an autoantigen bound to the MHC polypeptide, in biological samples. In some embodiments, the kits comprise a first container containing a first antibody capable of immunoreacting with a diversity region of a novel polypeptide disclosed herein, with the first antibody present in an amount sufficient to perform at least one assay. Preferably, the assay kits of the presently disclosed subject matter further comprise a second container containing a second antibody that immunoreacts with the first antibody. More preferably, the antibodies used in the assay kits of the presently disclosed subject matter are monoclonal antibodies. Even more preferably, the first antibody is affixed to a solid support. More preferably still, the first and second antibodies comprise an indicator, and, preferably, the indicator is a radioactive label or an enzyme.

A biological sample to be screened can be a biological fluid such as extracellular or intracellular fluid or a cell or tissue extract or homogenate. A biological sample can also be an isolated cell (e.g., in culture) or a collection of cells such as in a tissue sample or histology sample. A tissue sample can be suspended in a liquid medium or fixed onto a solid support such as a microscope slide.

In accordance with an exemplary use of a screening assay described above, a biological sample is exposed to an antibody immunoreactive with the polypeptide whose presence is being assayed. Typically, exposure is accomplished by forming an admixture in a liquid medium that contains both the antibody and the candidate polypeptide. Either the antibody or the sample with the polypeptide can be affixed to a solid support (e.g., a column or a microtiter plate).

The biological sample is exposed to the antibody under biological reaction conditions and for a period of time sufficient for antibody-polypeptide conjugate formation. Biological reaction conditions include ionic composition and concentration, temperature, pH and the like.

Ionic composition and concentration can range from that of distilled water to a 2 molal solution of NaCl. Preferably, osmolality is from about 100 mosmols/l to about 400 mosmols/l and, more preferably from about 200 mosmols/l to about 300 mosmols/l. Temperature preferably is from about 4° C. to about 100° C., more preferably from about 15° C. to about 50° C. and, even more preferably from about 25° C. to about 40° C. pH is preferably from about a value of 4.0 to a value of about 9.0, more preferably from about a value of 6.5 to a value of about 8.5 and, even more preferably from about a value of 7.0 to a value of about 7.5. The only limit on biological reaction conditions is that the conditions selected allow for antibody-polypeptide conjugate formation and that the conditions do not adversely affect either the antibody or the polypeptide.

Exposure time will vary inter alia with the biological conditions used, the concentration of antibody and polypeptide and the nature of the sample (e.g., fluid or tissue sample). Approaches for determining exposure time are well known to one of ordinary skill in the art. Typically, where the sample is fluid and the concentration of polypeptide in that sample is about 10⁻¹⁰M, exposure time is from about 10 minutes to about 200 minutes.

The presence of polypeptide in the sample is detected by detecting the formation and presence of antibody-polypeptide conjugates. Approaches for detecting such antibody-antigen (e.g., receptor polypeptide) conjugates or complexes are well known in the art and include such procedures as centrifugation, affinity chromatography and the like, binding of a secondary antibody to the antibody-candidate receptor complex.

In one embodiment, detection is accomplished by detecting an indicator affixed to the antibody. Exemplary and well known such indicators include radioactive labels (e.g., ³²P, ¹²⁵I, ¹⁴C), a second antibody or an enzyme such as horseradish peroxidase. Approaches for affixing indicators to antibodies are well known in the art. Commercial kits are available.

In another aspect of the presently disclosed subject matter, assay kits are provided for detecting the presence of an autoantigen bound to an MHC polypeptide, for example a human HLA-DR4 polypeptide, in a biological sample. In some embodiments, the autoantigen comprises a rheumatoid arthritis-associated polypeptide, such as for example gp39 or collagen II. In some embodiments, the kits comprise a first container containing a novel isolated TCR polypeptide disclosed herein. The kits can further comprise in some embodiments a reagent for detecting a complex comprising the isolated TCR polypeptide and the autoantigen bound to the MHC polypeptide. The reagent can be an indicator, which can be a radioactive label or an enzyme. In some embodiments, the TCR polypeptide is bound to a solid support.

The presently disclosed subject matter also provides a kit for screening compounds having binding affinity for a TCR polypeptide antigen-MHC binding site. The “antigen-MHC binding site” of a TCR polypeptide is a region of the polypeptide sequence necessary for affinity binding of the TCR polypeptide to the antigen-MHC complex. Such a kit can contain a first container containing an isolated novel TCR polypeptide disclosed herein. The kit can also contain one or more reagents for detecting a complex comprising the TCR polypeptide and a compound bound to the antigen-MHC binding site of the TCR polypeptide. The provided reagent can be an indicator, which can be radiolabeled or can be an enzyme. The TCR polypeptide can be bound to a solid support.

The reagents of the kits disclosed herein can be provided as a liquid solution, attached to a solid support or as a dried powder. Preferably, when the reagent is provided in a liquid solution, the liquid solution is an aqueous solution. Preferably, when the reagent provided is attached to a solid support, the solid support can be chromatograph media or a microscope slide. When the reagent provided is a dry powder, the powder can be reconstituted by the addition of a suitable solvent. The solvent can be provided.

IX. Screening Methods

The presently disclosed subject matter also provides methods of screening for candidate compounds having a binding affinity for a T cell receptor antigen-MHC binding site. In some embodiments, the method comprises providing a novel T cell receptor disclosed herein comprising an antigen-MHC binding-site, wherein the antigen is a human autoantigen; contacting the T cell receptor with a test compound; measuring for binding of the test compound to the antigen-MHC binding site of the T cell receptor; and selecting the test compound as a candidate compound if the test compound binds the antigen-MHC binding site of the T cell receptor.

A candidate compound identified according to the screening assay described herein would have the ability to bind with specificity a TCR at its antigen-MHC binding site, which is indicative of the candidate compound being capable of modulating the biological activity of the TCR, and/or inhibit the TCR from binding an antigen-MHC complex to which it has affinity. Since the antigen to which the TCR has specificity is a human autoantigen, identified candidate compounds that can bind the TCR at its antigen-MHC binding site can possibly disrupt the activation of T cells expressing the TCRs described herein by disrupting the binding of the TCR to the autoantigen-MHC complex presented on the surface of an antigen presenting cell. Such a candidate compound has utility in the treatment of autoimmune disorders, and in particular rheumatoid arthritis.

In some embodiments of the presently disclosed subject matter, the autoantigen is a rheumatoid arthritis-associated autoantigen, such as for example gp39 or collagen II. Further, in some embodiments, the antigen-MHC binding site is specific for an HLA-DR4 MHC polypeptide bound with an autoantigen, such as for example gp39 or collagen II. In some embodiments, the test compound is a polypeptide. Also, in some embodiments, the T cell receptor is bound to a substrate.

X. Diagnostic and Therapeutic Methods

As discussed herein, the presentation by specific classes of MHC molecules of certain autoantigens and the recognition of these autoantigen-MHC complexes on APCs by T cells has been associated with the development of rheumatoid arthritis. Without wishing to be bound by a particular theory of operation, it is believed recognition by the T cells of these autoantigens complexed with specific MHC class polypeptides results in activation of the T cells, which through a cascade of events, a variety of components of the immune system, resulting in inflammation, damage to connective tissue, and other symptoms associated with rheumatoid arthritis. Thus, the identification and characterization of novel TCRs disclosed herein, which have binding specificity for these MHC polypeptide classes and autoantigens presented by these MHC polypeptides known to be associated with rheumatoid arthritis, have utility as therapeutic and diagnostic tools.

For example, in some embodiments of the presently disclosed subject matter, the isolated novel TCR polypeptides can be used with methods of determining a subject's risk for developing rheumatoid arthritis. In another embodiment of the presently disclosed subject matter, the isolated novel TCR polypeptides can be used in methods of inhibiting binding of a TCR to an MHC polypeptide and an autoantigen bound to the MHC polypeptide in a subject. In a further embodiment of the presently disclosed subject matter, the isolated novel TCR polypeptides can be used in methods of eliciting an immune response against a TCR polypeptide having binding specificity for an MHC polypeptide and an autoantigen bound to the MHC polypeptide in a subject.

With respect to the therapeutic methods of the presently disclosed subject matter, any animal subject can be treated. The term “subject” as used herein refers to any vertebrate species. The methods of the presently claimed subject matter are particularly useful in the diagnosis of warm-blooded vertebrates. Thus, the presently claimed subject matter concerns mammals. More particularly contemplated is the diagnosis and/or treatment of mammals such as humans with autoimmune disorders, such as for example rheumatoid arthritis, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also contemplated is the diagnosis and/or treatment of autoimmune diseases, such as for example rheumatoid arthritis, in livestock, including, but not limited to domesticated swine (pigs and hogs), ruminants, horses, poultry, and the like.

In methods of the presently disclosed subject matter described below, wherein an amount of an isolated TCR polypeptide is administered to a subject, a therapeutically effective amount of the TCR polypeptide is typically an amount of polypeptide such that when administered in a physiologically tolerable composition is sufficient to achieve a plasma concentration of from about 0.001 microgram (μg) per milliliter (ml) to about 10 μg/ml, preferably from about 0.05 μg/ml to about 1.0 μg/ml.

The novel polypeptides described herein when given therapeutically can be administered parenterally by injection or by gradual infusion over time. Although the tissue to be treated can typically be accessed in the body by systemic administration and therefore most often treated by intravenous administration of therapeutic compositions, other tissues and delivery approaches are provided where there is a likelihood that the tissue targeted contains the target molecule. Thus, polypeptides of the presently disclosed subject matter can be administered intravenously, intraperitoneally, intramuscularly, subcutaneously, intra-cavity, transdermally, and can be delivered by peristaltic means.

The therapeutic compositions containing a polypeptide of subject matter disclosed herein are conventionally administered intravenously, as by injection of a unit dose, for example. The term “unit dose” when used in reference to a therapeutic composition of the presently disclosed subject matter refers to physically discrete units suitable as unitary dosage for the subject, each unit containing a predetermined quantity of active material calculated to produce the desired therapeutic effect in association with the required diluent; i.e., carrier or excipient.

The compositions are administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required for administration depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

X.A. Methods of Determining Risk of Developing Rheumatoid Arthritis

The presently disclosed subject matter further provides methods of determining a test subject's risk for developing rheumatoid arthritis. In some embodiments, the method comprises providing a novel T cell receptor polypeptide disclosed herein having binding affinity for a human autoantigen bound to an HLA-DR4 polypeptide; contacting a biological sample from a test subject with the T cell receptor polypeptide, wherein the biological sample comprises MHC polypeptides from the test subject; detecting binding of the MHC polypeptides with the T cell receptor polypeptide; and determining a test subject is at risk for developing rheumatoid arthritis if at least one of the MHC polypeptides binds the T cell receptor polypeptide. By determining one of a presence, amount, and both presence and amount of a TCR polypeptide disclosed herein, a risk for developing rheumatoid arthritis by the subject can be confirmed, since the identified TCR has been associated as a potential actor in the development and progression of rheumatoid arthritis.

In some embodiments of the methods, the human autoantigen is gp39 or collagen II. In some embodiments, the T cell receptor polypeptide is bound to a substrate. Further, in some embodiments, the biological sample comprises blood.

X.B. Methods of Modulating Binding of a TCR to an MHC-Antigen Complex

The presently disclosed subject matter further provides methods of inhibiting binding of a T cell receptor to an MHC polypeptide and an autoantigen bound to the MHC polypeptide in a subject. The method comprises administering to a subject an isolated T cell receptor polypeptide having binding specificity for an MHC polypeptide and an autoantigen bound to the MHC polypeptide. Administration to the subject of a novel isolated TCR disclosed herein can inhibit binding of the subject's own TCR polypeptides having specificity for the autoantigen bound to the MHC polypeptide, by for example competing with the subject's own TCR polypeptide, thus modulating the amount of activation of the subject's T cells and thereby alleviating symptoms of or slowing progression of rheumatoid arthritis in the subject.

In some embodiments, the T cell receptor polypeptide is in a pharmaceutically acceptable carrier. Further, in some embodiments, the T cell receptor polypeptide is soluble in an aqueous solution.

In some embodiments, the MHC polypeptide is HLA-DR4. In some embodiments, the autoantigen is gp39 or collagen II, and further can be human gp39 or human collagen II.

X.C. Methods of Eliciting an Immune Response Against a TCR

The presently disclosed subject matter further provides methods of eliciting an immune response against a T cell receptor polypeptide having binding specificity for an MHC polypeptide and an autoantigen bound to the MHC polypeptide in a subject. The method comprises administering to a subject a composition comprising an isolated T cell receptor polypeptide having binding specificity for an MHC polypeptide and an autoantigen bound to the MHC polypeptide. Administration to the subject of a novel isolated TCR polypeptide disclosed herein can lead to stimulation of the subject's immune system against the subject's own similar TCR polypeptides expressed by the subject's T cells.

In some embodiments, the composition further comprises an adjuvant, which can further enhance the production of an immune response against the subject's own homologous TCR polypeptides. Suitable exemplary adjuvants are discussed in detail herein at Section V. Production of an immune response can provide for blocking the subject's T cells from becoming activated by autoantigen by, for example, inhibiting binding of the TCR to the autoantigen-MHC complex or through removal of the T cell from circulation by the immune system.

In some embodiments, the MHC polypeptide is HLA-DR4. Further, in some embodiments, the autoantigen is gp39 or collagen II, and can be a human gp39 or human collagen II.

### EXAMPLES

### Example 1

### Production of the gp39 and hCII Specific T Cell Hybridoma Lines

As shown generally in FIG. 2, T cell hybridoma lines were generated by fusing activated T cells derived from an HLA-DR4 transgenic mouse immunized with either human gp39 or collagen II (CII). T cells were then fused with BW5147 cells (ATCC, Manassas, Va., U.S.A.) and stable hybridomas were selected for using HAT, using art-recognized techniques.

### Example 2

### Antigen Specificity Determination of T cell Hybridoma Cell Lines

Cell lines surviving HAT selection were screened for antigen specificity. Specificity of the T cells was tested using an antigen presentation assay to stimulate the T cells. Measurement of their IL-2 production by a bioassay using a cell line that is dependent on IL-2 for growth demonstrated evidence of their stimulation. Results are shown in Table 1 below. The DR4gp-32 line, which was produced from immunization with gp39 of the transgenic mouse expressing human HLA-DR4, only responds when the gp39 protein is present. In contrast, the DR4hCII-36 & 61 lines, which were produced from immunization with CII of the transgenic mouse expressing human HLA-DR4, only responds to CII.

TABLE 1
Antigen Specificity Of The T Cell Receptors
Expressed By The T Cell Hybridoma Lines

IL-2, U/mL

| T-Cell Line | Antigen | −Ag | +Ag |
|---|---|---|---|
| DR4gp-32 | gp39 | <20 | 640 |
| DR4hCII-36 | CII | <20 | 1280 |
| DR4hCII-61 | CII | <20 | 1280 |

The ability of the novel hybridoma T cell lines to be stimulated by their respective antigen, gp39 or CII, was further tested using two different antigen presenting cells expressing either HLA-DR1 (DRB1*0I01) or HLA-DR4 (DRB1*0401). Results are shown in Table 2. IL-2 production indicated stimulation of the T cells and was measured by bioassay as above. As demonstrated by the results in Table 2, the T cell receptors expressed by the novel T cell hybridoma cell lines disclosed herein only recognize their respective antigen when it is presented by the HLA-DR4 molecule.

TABLE 2
HLA Restriction of the gp39 and CII T Cell Hybridoma Lines

| | | IL-2, U/mL | |
|---|---|---|---|
| | | HLA-DR type of antigen presenting cell | |
| T-Cell Line | Antigen | DRB1*0101 (DR1) | DRB1*0401 (DR4) |
| DR4gp-32 | gp39 | <20 | 1280 |
| DR4hCII-36 | CII | <20 | 1280 |
| DR4hCII-61 | CII | <20 | <2560 |

### Example 3

### Isolation, Cloning and Sequencing of TCR polypeptides from T Cell Hybridomas

RNA was isolated from the T cell hybridomas, converted to cDNA, and amplified by PCR using panels of bet chain and alpha chain specific 5′ PCR primers and C region 3′ primers using techniques well known in the art. PCR products were then cloned and sequenced using techniques well known in the art.

Table 3 below identifies the DNA and polypeptide sequences (and SEQ ID NOs) of the TCR polypeptides derived from the T cell hybridoma cell lines.

TABLE 3
Cloned TCR Polypeptides

| SEQ | Antigen | |
|---|---|---|
| ID NO | Specificity | Sequence |
| 1 | CII | DR4hCII61 α chain DNA |
| 2 | CII | DR4hCII61 α chain peptide |
| 3 | CII | DR4hCII61 β chain DNA |
| 4 | CII | DR4hCll61 β chain peptide |
| 5 | CII | DR4hCII36 α chain DNA |
| 6 | CII | DR4hCII36 α chain peptide |
| 7 | CII | DR4hCII36 β chain DNA |
| 8 | CII | DR4hCII36 β chain peptide |
| 9 | gp39 | DR4gp32 α chain DNA |
| 10 | gp39 | DR4gp32 α chain peptide |
| 11 | gp39 | DR4gp32 β chain DNA |
| 12 | gp39 | DR4gp32 β chain peptide |

### REFERENCES

Adelman et al. (1983) DNA 2:183.
Alam & Cook (1990) Anal. Biochem. 188:245-254.
Altschul et al. (1990) J. Mol. Biol. 215:403-410.
Ausubel et al. (1992) Current Protocols in Molecular Biology (J. Wylie & Sons,
Barton G J (1998) Acta Crystallogr. D. Biol. Crystallogr. 54:1139-1146.
Batzer et al. (1991) Nucleic Acids Res. 19:5081.
Cubitt et al. (1995) Trends Biochem. Sci. 20:448-455.
Glover & Hames (1995) DNA Cloning: A Practical Approach, 2nd ed. IRL Press at Oxford University Press, Oxford; N.Y.
Greenberg et al. (1994) Mol. Endocrinol. 8:230-239.
Henikoff et al. (2000) Electrophoresis 21:1700-1706.
Henikoff & Henikoff (2000) Adv. Protein Chem. 54:73-97.
Howell et al. (1988) Antibodies A Laboratory Manual, (Cold Spring Harbor Laboratory).
Huang (2000) Pac. Symp. Biocomput. 230-241.
Karlin & Altschul (1993) Proc Natl Acad Sci USA 90:5873-5877.
Kurihara et al. (2000) J Clin Invest 106:763-771.
Kyte et al. (1982) J. Mol. Biol. 157:105.
Lee (2000) Anticancer Res 20:417-422.
Needleman et al. (1970) J. Mol. Biol. 48:443.
Ohtsuka et al. (1985) J Biol Chem 260:2605-2608.
Pearson & Lipman (1988) Proc Natl Acad Sci USA 85:2444-2448.
Rose & Botstein (1983) Meth Enzymol 101:167-180.
Rossolini et al. (1994) Mol Cell Probes 8:91-98.
Sambrook et al. (2001) Molecular Cloning: A Laboratory Manual (Cold Spring Harbor Laboratory Press, Cold Spring Harbor, N.Y.).
Saqi et al. (1999) Bioinformatics 15:521-522.
Scharfmann et al. (1991) Proc Natl Acad Sci USA 88:4626-4630.
Silhavy et al. (1984) Experiments with Gene Fusions. Cold Spring Harbor Laboratory, Cold Spring Harbor, N.Y.
Smith & Waterman, Adv. Appl. Math. 2:482 (1981).
Tijssen (1993) Laboratory Techniques in Biochemistry and Molecular Biology-Hybridization with Nucleic Acid Probes, part I chapter 2, Elsevier, New York, N.Y.
U.S. Pat. No. 4,196,265
U.S. Pat. No. 4,554,101
U.S. Pat. No. 4,736,866
U.S. Pat. No. 5,162,215
U.S. Pat. No. 5,489,742
U.S. Pat. No. 5,550,316
U.S. Pat. No. 5,573,933
U.S. Pat. No. 5,614,396
U.S. Pat. No. 5,625,125
U.S. Pat. No. 5,648,061
U.S. Pat. No. 5,741,957
Williams et al.(1993) J Clin Invest 92:503-508.
WO 97/47763
Yu et al. (1999) Cancer Res 59:4200-4203.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. An isolated T cell receptor polypeptide having binding specificity for an MHC polypeptide and an autoantigen bound to the MHC polypeptide.

2. The T cell receptor polypeptide of claim 1, wherein the MHC polypeptide is HLA-DR4.

3. The T cell receptor polypeptide of claim 2, wherein the autoantigen is gp39 or collagen II.

4. The T cell receptor polypeptide of claim 3, wherein the autoantigen is a human gp39 or human collagen II.

5. The T cell receptor polypeptide of claim 1, wherein the T cell receptor polypeptide is a heterodimeric polypeptide comprising α chain and a β chain.

6. The T cell receptor polypeptide of claim 5, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a fragment of a polypeptide of a), b), c), d), or e).

7. The T cell receptor polypeptide of claim 5, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a portion of a polypeptide of a), b), c), d), or e).

8. An isolated nucleic acid molecule encoding a T cell receptor polypeptide having binding specificity for an MHC polypeptide and an autoantigen bound to the MHC polypeptide.

9. The isolated nucleic acid molecule of claim 8, wherein the MHC polypeptide is HLA-DR4.

10. The isolated nucleic acid molecule of claim 8, wherein the autoantigen is gp39 or collagen II.

11. The isolated nucleic acid molecule of claim 10, wherein the autoantigen is a human gp39 or human collagen II.

12. The isolated nucleic acid molecule of claim 8, wherein the encoded polypeptide comprises an amino acid sequence of any of SEQ ID NOs: 2, 4, 6, 8, 10 and 12.

13. The isolated nucleic acid molecule of claim 8, wherein the encoded polypeptide is a heterodimeric polypeptide comprising α chain and a β chain.

14. The isolated nucleic acid molecule of claim 13, wherein the α chain has an amino acid sequence of any of SEQ ID NOs:2, 6 and 10, or a biologically functional equivalent thereof and the β chain has an amino acid sequence of any of SEQ ID NOs:4, 8 and 12.

15. A chimeric gene, comprising the nucleic acid molecule of claim 8 operably linked to a heterologous promoter.

16. A vector comprising the chimeric gene of claim 15.

17. A host cell comprising the vector of claim 16.

18. An isolated antibody capable of specifically binding to a diversity region of a polypeptide of claim 1.

19. The antibody of claim 18, wherein the antibody is capable of modulating the biological activity of an isolated T cell receptor polypeptide having binding specificity for an MHC polypeptide and an autoantigen bound to the MHC polypeptide.

20. A hybridoma cell line which produces the antibody of claim 18.

21. A method of detecting a nucleic acid molecule that encodes a T cell receptor polypeptide having binding specificity for an MHC polypeptide and an autoantigen bound to the MHC polypeptide in a biological sample containing nucleic acid material, the method comprising:
(a) hybridizing the nucleic acid molecule of claim 8 under stringent hybridization conditions to the nucleic acid material of the biological sample, thereby forming a hybridization duplex; and
(b) detecting the hybridization duplex.

22. The method of claim 21, wherein the MHC polypeptide is HLA-DR4.

23. The method of claim 22, wherein the autoantigen is gp39 or collagen II.

24. The method of claim 23, wherein the autoantigen is a human gp39 or collagen II.

25. A T cell hybridoma, comprising a T cell receptor polypeptide having binding specificity for an MHC polypeptide and an autoantigen bound to the MHC polypeptide.

26. The T cell hybridoma of claim 25, wherein the MHC polypeptide is HLA-DR4.

27. The T cell hybridoma of claim 25, wherein the autoantigen is gp39 or collagen II.

28. The T cell hybridoma of claim 27, wherein the autoantigen is a human gp39 or human collagen II.

29. The T cell hybridoma of claim 25, wherein the polypeptide comprises an amino acid sequence of any of SEQ ID NOs: 2, 4, 6, 8, 10 and 12.

30. The T cell hybridoma of claim 25, wherein the polypeptide is a heterodimeric polypeptide comprising α chain and a β chain.

31. The T cell hybridoma of claim 30, wherein the α chain has an amino acid sequence of any of SEQ ID NOs:2, 6 and 10, or a biologically functional equivalent thereof and the β chain has an amino acid sequence of any of SEQ ID NOs:4, 8 and 12 or a biologically functional equivalent thereof.

32. A transgenic or chimeric non-human animal, comprising:
(a) a polynucleic acid encoding a biologically active human MHC polypeptide which is present in the genome in a copy number effective to confer expression in the animal of the human MHC polypeptide; and
(b) a human rheumatoid arthritis-associated polypeptide in an amount sufficient to induce production by the mouse of activated T cells expressing a T cell receptor with binding specificity for the human rheumatoid arthritis-associated polypeptide bound to the expressed human MHC polypeptide.

33. The transgenic or chimeric non-human animal of claim 32, wherein the human MHC polypeptide is HLA-DR4.

34. The transgenic or chimeric non-human animal of claim 32, wherein the human rheumatoid arthritis-associated polypeptide is gp39 or collagen II.

35. The transgenic or chimeric non-human animal of claim 32, wherein the T cell receptor is a heterodimeric polypeptide comprising α chain and a β chain.

36. The transgenic or chimeric non-human animal of claim 35, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a portion of a polypeptide of a), b), c), d), or e).

37. The transgenic or chimeric non-human animal of claim 35, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a portion of a polypeptide of a), b), c), d), or e).

38. The transgenic or chimeric non-human animal of claim 32, wherein the non-human animal is a mouse.

39. An assay kit for detecting the presence of a T cell receptor polypeptide having binding specificity for an MHC polypeptide and an autoantigen bound to the MHC polypeptide in a biological sample, the kit comprising a first container containing a first antibody capable of immunoreacting with a diversity region of a polypeptide of claim 1, wherein the first antibody is present in an amount sufficient to perform at least one assay.

40. The assay kit of claim 39, further comprising a second container containing a second antibody that immunoreacts with the first antibody.

41. The assay kit of claim 40, wherein the first antibody and the second antibody comprise monoclonal antibodies.

42. The assay kit of claim 39, wherein the first antibody is affixed to a solid support.

43. The assay kit of claim 39, wherein the first and second antibodies each comprise an indicator.

44. The assay kit of claim 43, wherein the indicator is a radioactive label or an enzyme.

45. The assay kit of claim 39, wherein the MHC polypeptide is HLA-DR4.

46. The assay kit of claim 39, wherein the autoantigen is gp39 or collagen II.

47. The assay kit of claim 46, wherein the autoantigen is a human gp39 or human collagen II.

48. An assay kit for detecting the presence of an autoantigen bound to an HLA-DR4 polypeptide in a biological sample, the kit comprising a first container containing an isolated T cell receptor polypeptide of claim 1.

49. The assay kit of claim 48, further comprising a reagent for detecting a complex comprising the isolated T cell receptor polypeptide and the autoantigen bound to the HLA-DR4 polypeptide.

50. The assay kit of claim 49, wherein the reagent is an indicator.

51. The assay kit of claim 50, wherein the indicator is a radioactive label or an enzyme.

52. The assay kit of claim 48, wherein the isolated T cell receptor polypeptide is bound to a solid support.

53. The assay kit of claim 49, wherein the autoantigen is gp39 or collagen II.

54. An assay kit for screening compounds having binding affinity for a T cell receptor antigen-MHC binding site, the kit comprising a first container containing an isolated T cell receptor polypeptide of claim 1.

55. The assay kit of claim 54, further comprising a reagent for detecting a complex comprising the isolated T cell receptor and a compound bound to the antigen-MHC binding site of the isolated T cell receptor polypeptide.

56. The assay kit of claim 55, wherein the reagent is an indicator.

57. The assay kit of claim 56 wherein the indicator is a radioactive label or an enzyme.

58. The assay kit of claim 54, wherein the isolated T cell receptor polypeptide is bound to a solid support.

59. A method of producing a T cell hybridoma which expresses a T cell receptor polypeptide having binding specificity for a human autoantigen bound to an HLA-DR4 polypeptide, the method comprising:
(a) immunizing a transgenic or chimeric non-human animal with a human autoantigen, wherein the non-human animal expresses an HLA-DR4 polypeptide;
(b) isolating a T cell activated by the human autoantigen from the non-human animal; and
(c) producing a T cell hybridoma by fusing the isolated activated T cell with an immortal cell, wherein the T cell hybridoma expresses a T cell receptor polypeptide having binding specificity for the human autoantigen bound to the HLA-DR4 polypeptide.

60. The method of claim 59, further comprising:
(d) isolating the T cell hybridoma expressing the T cell receptor having binding specificity for the human autoantigen bound to the HLA-DR4 polypeptide.

61. The method of claim 59, wherein the human autoantigen is gp39 or collagen II.

62. The method of claim 59, wherein the non-human animal is a mouse.

63. The method of claim 59, wherein the immortal cell is a BW5147 cell.

64. The method of claim 59, wherein the T cell receptor polypeptide is an isolated T cell receptor polypeptide having binding specificity for an MHC polypeptide and an autoantigen bound to the MHC polypeptide.

65. The method of claim 64, wherein the T cell receptor polypeptide is a heterodimeric polypeptide comprising α chain and a β chain.

66. The method of claim 65, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a portion of a polypeptide of a), b), c), d), or e).

67. The method of claim 65, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a fragment of a polypeptide of a), b), c), d), or e).

68. A method of screening for candidate compounds having a binding affinity for a T cell receptor antigen-MHC binding site, the method comprising:
(a) providing a T cell receptor comprising an antigen-MHC binding site, wherein the antigen is a human autoantigen;
(b) contacting the T cell receptor with a test compound;
(c) measuring for binding of the test compound to the antigen-MHC binding site of the T cell receptor; and
(d) selecting the test compound as a candidate compound if the test compound binds the antigen-MHC binding site of the T cell receptor.

69. The method of claim 68, wherein the human autoantigen is gp39 or collagen II.

70. The method of claim 69, wherein the antigen-MHC binding site is specific for HLA-DR4 bound with gp39 or collagen II.

71. The method of claim 68, wherein the test compound is a polypeptide.

72. The method of claim 68, wherein the T cell receptor is bound to a substrate.

73. The method of claim 68, wherein the T cell receptor polypeptide is an isolated T cell receptor polypeptide having binding specificity for an MHC polypeptide and an autoantigen bound to the MHC polypeptide.

74. The method of claim 73, wherein the T cell receptor polypeptide is a heterodimeric polypeptide comprising α chain and a β chain.

75. The method of claim 74, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a portion of a polypeptide of a), b), c), d), or e).

76. The method of claim 74, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a fragment of a polypeptide of a), b), c), d), or e).

77. A method of determining a test subject's risk for developing rheumatoid arthritis, the method comprising:
(a) providing a T cell receptor polypeptide having binding affinity for a human autoantigen bound to an HLA-DR4 polypeptide;
(b) contacting a biological sample from a test subject with the T cell receptor polypeptide, wherein the biological sample comprises MHC polypeptides from the test subject;
(c) detecting binding of the MHC polypeptides with the T cell receptor polypeptide; and
(d) determining a test subject is at risk for developing rheumatoid arthritis if at least one of the MHC polypeptide binds the T cell receptor polypeptide.

78. The method of claim 77, wherein the human autoantigen is gp39 or collagen II.

79. The method of claim 77, wherein the T cell receptor polypeptide is bound to a substrate.

80. The method of claim 77, wherein the biological sample comprises blood.

81. The method of claim 77, wherein the T cell receptor polypeptide is an isolated T cell receptor polypeptide having binding specificity for an MHC polypeptide and an autoantigen bound to the MHC polypeptide.

82. The method of claim 81, wherein the T cell receptor polypeptide is a heterodimeric polypeptide comprising α chain and a β chain.

83. The method of claim 82, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a portion of a polypeptide of a), b), c), d), or e).

84. The method of claim 82, wherein the D chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a portion of a polypeptide of a), b), c), d), or e).

85. A method of inhibiting binding of a T cell receptor to an MHC polypeptide and an autoantigen bound to the MHC polypeptide in a subject, the method comprising administering to a subject an isolated T cell receptor polypeptide having binding specificity for an MHC polypeptide and an autoantigen bound to the MHC polypeptide.

86. The method of claim 85, wherein the T cell receptor polypeptide is in a pharmaceutically acceptable carrier.

87. The method of claim 85, wherein the T cell receptor polypeptide is soluble in an aqueous solution.

88. The method of claim 85, wherein the MHC polypeptide is HLA-DR4.

89. The method of claim 86, wherein the autoantigen is gp39 or collagen II.

90. The method of claim 89, wherein the autoantigen is a human gp39 or human collagen II.

91. The method of claim 85, wherein the T cell receptor polypeptide is a heterodimeric polypeptide comprising α chain and a β chain.

92. The method of claim 91, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:l, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a fragment of a polypeptide of a), b), c), d), or e).

93. The method of claim 91, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a portion of a polypeptide of a), b), c), d), or e).

94. A method of eliciting an immune response against a T cell receptor polypeptide having binding specificity for an MHC polypeptide and an autoantigen bound to the MHC polypeptide in a subject, the method comprising administering to a subject a composition comprising an isolated T cell receptor polypeptide having binding specificity for an MHC polypeptide and an autoantigen bound to the MHC polypeptide.

95. The method of claim 94, wherein the composition further comprises an adjuvant.

96. The method of claim 94, wherein the MHC polypeptide is HLA-DR4.

97. The method of claim 96, wherein the autoantigen is gp39 or collagen II.

98. The method of claim 97, wherein the autoantigen is a human gp39 or human collagen II.

99. The method of claim 94, wherein the T cell receptor polypeptide is a heterodimeric polypeptide comprising α chain and a β chain.

100. The method of claim 99, wherein the α chain comprises:
(a) a polypeptide encoded by a nucleic acid sequence as set forth in any of SEQ ID NOs:1, 5 and 9;
(b) a polypeptide encoded by a nucleic acid having at least about 70% or greater sequence identity to a DNA sequence as set forth in any of SEQ ID NOs:1, 5 and 9;
(c) a polypeptide encoded by a nucleic acid capable of hybridizing under stringent conditions to a nucleic acid comprising a sequence or the complement of a sequence as set forth in any of SEQ ID NOs:1, 5 and 9;
(d) a polypeptide having an amino acid sequence of any of SEQ ID NOs:2, 6 and 10, or a biologically functional equivalent thereof;
(e) a polypeptide which is immunologically cross-reactive with antibodies which are immunologically reactive with a diversity region of a polypeptide having an amino acid sequence of any of SEQ ID NOs:2, 6 and 10; or
(f) a polypeptide comprising a fragment of a polypeptide of a), b), c), d), or e).

101. The method of claim 99, wherein the β chain comprises:
(a) a polypeptide encoded by a nucleic acid sequence as set forth in any of SEQ ID NOs:3, 7 and 11;
(b) a polypeptide encoded by a nucleic acid having at least about 70% or greater sequence identity to a DNA sequence as set forth in any of SEQ ID NOs:3, 7 and 11;
(c) a polypeptide encoded by a nucleic acid capable of hybridizing under stringent conditions to a nucleic acid comprising a sequence or the complement of a sequence as set forth in any of SEQ ID NOs:3, 7 and 11;
(d) a polypeptide having an amino acid sequence of any of SEQ ID NOs:4, 8 and 12, or a biologically functional equivalent thereof;
(e) a polypeptide which is immunologically cross-reactive with antibodies which are immunologically reactive with a diversity region of a polypeptide having an amino acid sequence of any of SEQ ID NOs:4, 8 and 12; or
(f) a polypeptide comprising a portion of a polypeptide of a), b), c), d), or e).

102. A composition comprising the polypeptide of claim 1 and a carrier.


SEQUENCE LISTING (12 sequences)

SEQ ID NO: 1 - DNA, 606 bp, Artificial Sequence
  Alpha chain DNA sequence from a T cell receptor isolated and cloned from a T lymphocyte hybridoma originally produced using a T lymphocyte isolated from a transgenic mouse constitutively expressing human MHC molecules and immunized with a human antigen
caacagaaggtgcagcagagcccagaatccctcattgttccagagggaggcatggcctctctcaactgcacttccagtgatcgtaatgttgactacttctggtggtacagacagcactctgggaaaagccccaagatgctgatgtctatcttctccaatggtgaaaaggaagaaggcagattcacagttcacctcaataaagccagcctgcatacttccctgcacatcagagactcccagcccagtgactctgctctctacctctgtgcagcaagcatgggaggaagcaatgcaaagctaaccttcgggaaaggcactaaactctctgttaaatcaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccc

SEQ ID NO: 2 - protein, 202 aa, Artificial Sequence
  Alpha chain peptide sequence from a T cell receptor isolated and cloned from a T lymphocyte hybridoma originally produced using a T lymphocyte isolated from a transgenic mouse constitutively expressing human MHC molecules and immunized with a human antigen
QQKVQQSPESLIVPEGGMASLNCTSSDRNVDYFWWYRQHSGKSPKMLMSIFSNGEKEEGRFTVHLNKASLHTSLHIRDSQPSDSALYLCAASMGGSNAKLTFGKGTKLSVKSNIQNPEPAVYQLKDPRSQDSTLCLFTDFDSQINVPKTMESGTFITDKTVLDMKAMDSKSNGAIAWSNQTSFTCQDIFKETNATYPSSDVP

SEQ ID NO: 3 - DNA, 720 bp, Artificial Sequence
  Beta chain DNA sequence from a T cell receptor isolated and cloned from a T lymphocyte hybridoma originally produced using a T lymphocyte isolated from a transgenic mouse constitutively expressing human MHC molecules and immunized with a human antigen
gctcagactatccatcaatggccagtcgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagtacggagaagctgcttctcagccactctggcttctacctctgtgcctggagtctatcccttaaccaagacacccagtactttgggccaggcactcggctcctcgtgttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgt

SEQ ID NO: 4 - protein, 240 aa, Artificial Sequence
  Beta chain peptide sequence from a T cell receptor isolated and cloned from a T lymphocyte hybridoma originally produced using a T lymphocyte isolated from a transgenic mouse constitutively expressing human MHC molecules and immunized with a human antigen
AQTIHQWPVAEIKAVGSPLSLGCTIKGKSSPNLYWYWQATGGTLQQLFYSITVGQVESVVQLNLSASRPKDDQFILSTEKLLLSHSGFYLCAWSLSLNQDTQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADC

SEQ ID NO: 5 - DNA, 606 bp, Artificial Sequence
  Alpha chain DNA sequence from a T cell receptor isolated and cloned from a T lymphocyte hybridoma originally produced using a T lymphocyte isolated from a transgenic mouse constitutively expressing human MHC molecules and immunized with a human antigen
ggagactcagtgacccagaaggaaggcctggtcactctcaccgaggggttgcctgtgatgctgaactgcacctatcagactatttactcaaatgctttccttttctggtatgtgcactatctcaatgaatcccctcggctactcctgaagagctccacagacaacaagaggaccgagcaccaagggttccacgccactctccataagagcagcagctccttccatctgcagaagtcctcagcgcagctgtcagactctgccctgtactactgtgctccctccagtagcaactatcagttgatctggggctctgggaccaagctaattataaagccagacatccagaacccagaacctgctgtgtaccagttaaaagatcctcggtctcaggacagcaccctctgcctgttcaccgactttgactcccaagtcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccc

SEQ ID NO: 6 - protein, 202 aa, Artificial Sequence
  Alpha chain peptide sequence from a T cell receptor isolated and cloned from a T lymphocyte hybridoma originally produced using a T lymphocyte isolated from a transgenic mouse constitutively expressing human MHC molecules and immunized with a human antigen
GDSVTQKEGLVTLTEGLPVMLNCTYQTIYSNAFLFWYVHYLNESPRLLLKSSTDNKRTEHQGFHATLHKSSSSFHLQKSSAQLSDSALYYCAPSSSNYQLIWGSGTKLIIKPDIQNPEPAVYQLKDPRSQDSTLCLFTDFDSQVNVPKTMESGTFITDKTVLDMKAMDSKSNGAIAWSNQTSFTCQDIFKETNATYPSSDVP

SEQ ID NO: 7 - DNA, 720 bp, Artificial Sequence
  Beta chain DNA sequence from a T cell receptor isolated and cloned from a T lymphocyte hybridoma originally produced using a T lymphocyte isolated from a transgenic mouse constitutively expressing human MHC molecules and immunized with a human antigen
gctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtctaatggttaaccaagacacccagtactttgggccaggcactcggctcctcgtgttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgt

SEQ ID NO: 8 - protein, 240 aa, Artificial Sequence
  Beta chain peptide sequence from a T cell receptor isolated and cloned from a T lymphocyte hybridoma originally produced using a T lymphocyte isolated from a transgenic mouse constitutively expressing human MHC molecules and immunized with a human antigen
AQTIHQWPVAEIKAVGSPLSLGCTIKGKSSPNLYWYWQATGGTLQQLFYSITVGQVESVVQLNLSASRPKDDQFILSTEKLLLSHSGFYLCAWSLMVNQDTQYFGPGTRLLVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADC

SEQ ID NO: 9 - DNA, 609 bp, Artificial Sequence
  Alpha chain DNA sequence from a T cell receptor isolated and cloned from a T lymphocyte hybridoma originally produced using a T lymphocyte isolated from a transgenic mouse constitutively expressing human MHC molecules and immunized with a human antigen
ggagactcagtgacccagaaggaaggcctggtcactctcaccgaggggttgcctgtgatgctgaactgcacctatcagactatttactcaaatgctttccttttctggtatgtgcactatctcaatgaatcccctcggctactcctgaagagctccacagacaacaagaggaccgagcaccaagggttccacgccactctccataagagcagcagctccttccatctgcagaagtcctcagcgcagctgtcagactctgccctgtactactgtgctctgagccggaatagcaataacagaatcttctttggtgatgggacgcagctggtggtgaagcccaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccc

SEQ ID NO: 10 - protein, 203 aa, Artificial Sequence
  Alpha chain peptide sequence from a T cell receptor isolated and cloned from a T lymphocyte hybridoma originally produced using a T lymphocyte isolated from a transgenic mouse constitutively expressing human MHC molecules and immunized with a human antigen
GDSVTQKEGLVTLTEGLPVMLNCTYQTIYSNAFLFWYVHYLNESPRLLLKSSTDNKRTEHQGFHATLHKSSSSFHLQKSSAQLSDSALYYCALSRNSNNRIFFGDGTQLVVKPNIQNPEPAVYQLKDPRSQDSTLCLFTDFDSQINVPKTMESGTFITDKTVLDMKAMDSKSNGAIAWSNQTSFTCQDIFKETNATYPSSDVP

SEQ ID NO: 11 - DNA, 717 bp, Artificial Sequence
  Beta chain DNA sequence from a T cell receptor isolated and cloned from a T lymphocyte hybridoma originally produced using a T lymphocyte isolated from a transgenic mouse constitutively expressing human MHC molecules and immunized with a human antigen
gagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctataggagggactccgactacaccttcggctcagggaccaggcttttggtaatagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgt

SEQ ID NO: 12 - protein, 239 aa, Artificial Sequence
  Beta chain peptide sequence from a T cell receptor isolated and cloned from a T lymphocyte hybridoma originally produced using a T lymphocyte isolated from a transgenic mouse constitutively expressing human MHC molecules and immunized with a human antigen
ETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSYRRDSDYTFGSGTRLLVIEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADC